(19) United States
(12) Patent Application Publication
(10) Pub. No.: US 2007/0198054 A1
(43) Pub. Date: Aug. 23, 2007

(54) Acupressure tool

(76) Inventors: Jules Steven Hecht, New York, NY (US); Roberta Gilston-Hecht, New York, NY (US)

Correspondence Address: LITMAN LAW OFFICES, LTD., P.O. BOX 15035, CRYSTAL CITY STATION, ARLINGTON, VA, 22215, US

(21) Appl. No.: 11/356,233

(22) Filed: Feb. 17, 2006

(51) Int. Cl. A61B 17/00

(52) U.S. Cl. 606/204

(57) ABSTRACT

The acupressure tool is a therapeutic device for stimulating acupressure pressure points in a user's wrist. The acupressure tool includes a gripping handle portion, which is adapted for grasping by a first hand of the user. A pair of pressure-applying nodes are mounted on a lower surface of the gripping handle portion and project downwardly therefrom. The pressure-applying nodes are spaced apart so that when the user grasps the gripping handle portion in his or her first hand, the pressure-applying nodes may be positioned against the wrist of the user's second hand to simultaneously contact and stimulate a pair of acupressure pressure points in the user's wrist.

### BACKGROUND OF THE INVENTION

1. Field of the Invention

The present invention relates to devices for relieving pain, and particularly to an acupressure tool for relieving back pain by applying pressure to two pressure points in the wrist.

2. Description of the Related Art

Lower back pain is a common affliction that affects millions of people throughout the world. Such back pain may be chronic or acute. Conventional medical therapy may involve various forms of treatment, including medications (analgesics, muscle relaxants, etc.), physical therapy, bed rest, back braces, surgery, nerve stimulators (for example, a TENS stimulator), chiropractic manipulation, massage therapy, and various other treat modalities. Sometimes various combinations of conventional treatment are effective, but quite often the patient is unable to gain complete relief from low back pain and must seek out alternative remedies.

Acupressure is a traditional Chinese therapeutic technique, which, like acupuncture, relies on contacting and stimulating specific points on the body. Acupressure traditionally uses the fingers, thumbs, knuckles or elbows to apply gentle pressure to key points on the human body. According to the teachings of acupressure, applying pressure to appropriate “trigger points” on the body for a sufficient period of time is one technique for relieving pain that may be referred to another part of the body.

However, it may be difficult to locate a qualified practitioner of the art of acupressure. Moreover, many individuals are unable to locate authoritative texts on acupressure, or if they do, are unable to comprehend what they read sufficiently in order to locate the trigger points on their own body, or to know how to apply the appropriate pressure. Acupressure views the body's energy, or Qi, as flowing through twelve channels (also referred to as meridians), each of which is linked to a particular organ. There are about 365 pressure points located on the major channels, and about 650 individual pressure points. Locating the appropriate pressure points for the relief of lower back pain with precision can be a problem.

Therefore, there is a need for an acupressure device that can be used simply and easily by the layman to apply pressure to trigger points of the human body for the relief of lower back pain in accordance with the teachings of acupressure. Thus, an acupressure tool solving the aforementioned problems is desired.

### SUMMARY OF THE INVENTION

The acupressure tool is a therapeutic device for applying pressure to acupressure pressure points in a user's wrist for the relief of lower back pain. The acupressure tool includes a gripping handle portion, which is adapted for grasping by a first hand of the user. The gripping handle portion includes an upper surface, a lower surface and first and second opposed side surfaces. A pair of pressure-applying nodes are mounted on the lower surface of the gripping handle portion and project downwardly therefrom. The lower surface and the upper surface may have substantially arcuate cross-sectional contours. The first side surface may be provided with a recess for receiving at least one finger of the user's first hand. The second side surface may have a recess for receiving the user's thumb.

The pressure-applying nodes are spaced apart so that when the user grasps the gripping handle portion in one hand, the pressure-applying nodes may be positioned against the wrist of the user's other hand to simultaneously contact and stimulate a pair of acupressure pressure points in the user's wrist. The pressure-applying nodes may include rounded contact surfaces for comfortably contacting the user's wrist.

These and other features of the present invention will become readily apparent upon further review of the following specification and drawings.

### BRIEF DESCRIPTION OF THE DRAWINGS

FIG. 1 is an environmental, perspective view of an acupressure tool according to the present invention.

FIG. 2 is a side view illustrating a first side of the acupressure tool according to the present invention.

FIG. 3 is a side view illustrating a second side of the acupressure tool according to the present invention.

FIG. 4 is a bottom view of the acupressure tool according to the present invention.

Similar reference characters denote corresponding features consistently throughout the attached drawings.

### DETAILED DESCRIPTION OF THE PREFERRED EMBODIMENT

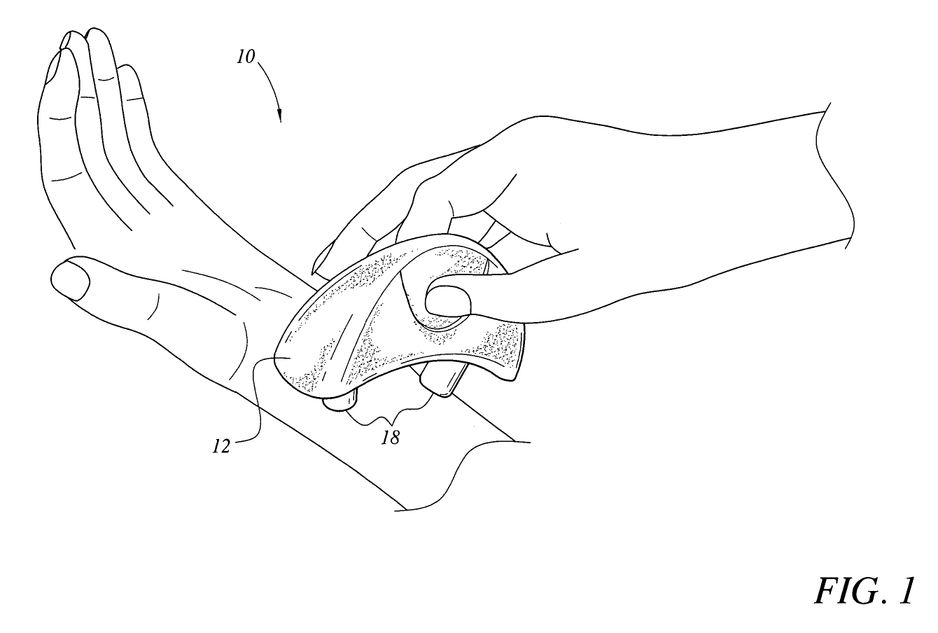

FIG. 1 illustrates a user stimulating acupressure pressure points on the user's wrist with an acupressure tool 10. Acupressure is a therapeutic technique involving the contact and stimulation of pressure points at specific locations on the human body. Acupressure tool 10 includes a gripping handle portion 12 having a pair of pressure-applying nodes 18 projecting downwardly therefrom, allowing the user to grasp the tool 10 with one hand while gently pressing the pressure-applying nodes 18 on the wrist of the user's other hand. Although shown as being used for self-application in FIG. 1, it should be understood that the acupressure tool 10 may be used by the user on another person.

Acupressure tool 10 is used for the relief of lower back pain and the relief of stiffness in the lower back. Practitioners of acupressure believe that applying pressure to “trigger” points in one part of the body can cause an increase in oxygenation of the muscles of another part of the body. As shown in FIG. 1, isometric pressure is applied to a pair of trigger points in the wrist of the user in order to provide relief from lower back pain. One or two applications of tool 10 to the wrist, each lasting one minute or less, may provide pain relief for several hours.

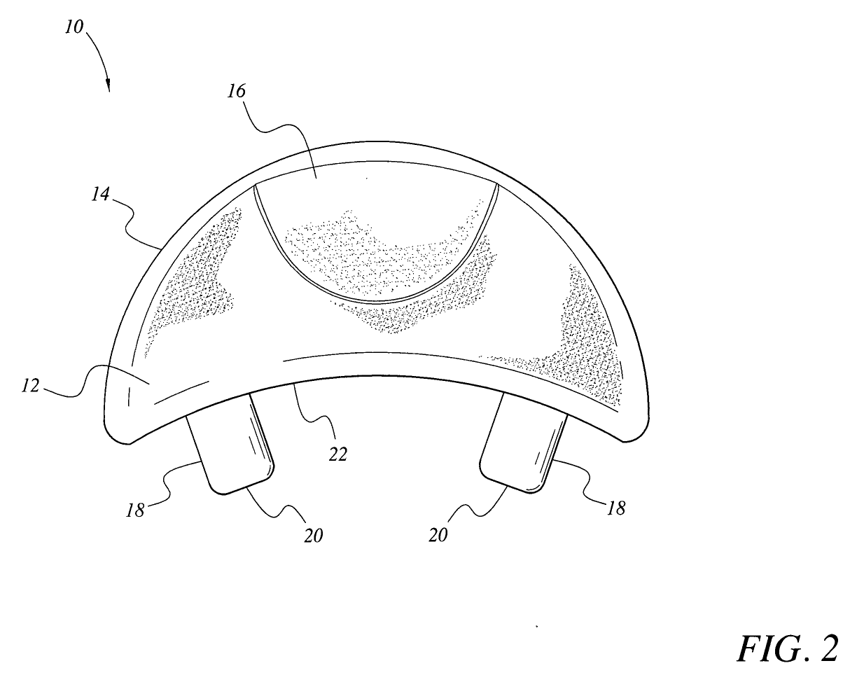

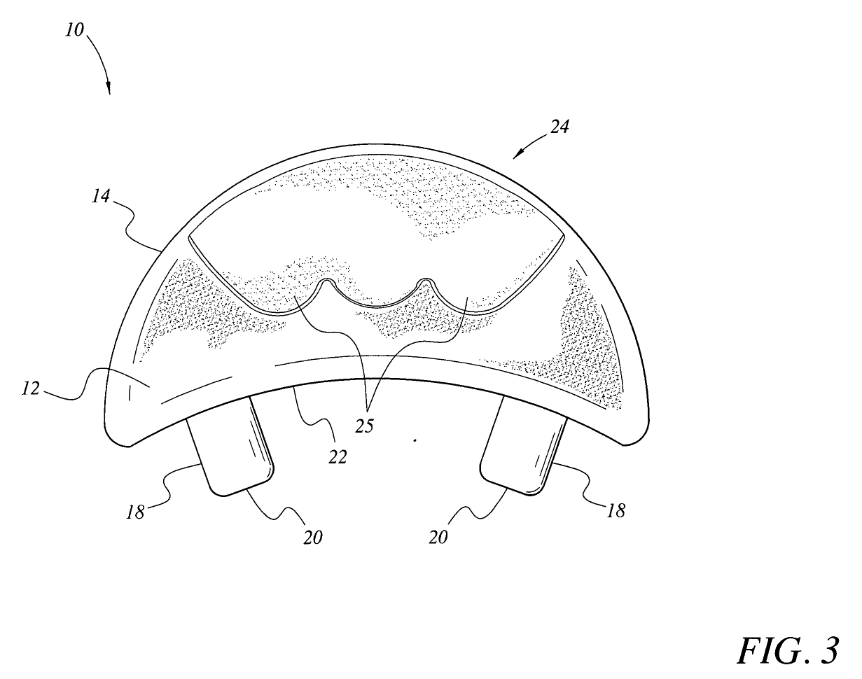

As best shown in FIGS. 2 and 3, the gripping handle portion 12 includes an upper surface 14, which preferably has a substantially arcuate cross-sectional contour, allowing the gripping handle portion 12 to be comfortably received and held within the user's hand. The upper surface 14 may have a substantially semicircular cross-sectional contour. However, upper surface 14 may have any suitable contour that provides a firm, comfortable grip, depending upon the needs and desires of the user.

Similarly, the lower surface 22 of gripping handle portion 12 also has a substantially arcuate cross-sectional contour. Lower surface 22 is preferably contoured to match the curve of the user's wrist, and the front surface may also be contoured to abut the base of the palm of the hand in order to properly position the pressure-applying nodes 18. The gripping handle portion 12 is preferably made from a transparent plastic material, such as acrylic or Plexiglass (Plexiglas is a trademark of Rohm & Hass, Co.), so that the user can visualize application of the nodes 18 to the trigger points during use. However, any suitable material that is comfortable for use by the user may be utilized. The gripping handle 12 may be an at least partially hollow body for lighter weight.

The pair of pressure-applying nodes 18 is mounted on lower surface 22 and project downwardly therefrom. Pressure-applying nodes 18 extend in a direction substantially normal to lower surface 22. The nodes 18 have a precise diameter and a precise spacing between the nodes in order to properly apply pressure to the trigger points in the wrist for users having a wide variety of physical builds. Pressure-applying nodes 18 are formed from rubber, either natural rubber or synthetic rubber, e.g., neoprene, of sufficient density and hardness to apply pressure to the trigger points, but having sufficient surface softness or resilience to be relatively comfortable as compared to rigid plastic.

Each node 18 is approximately ¼ to ½ of an inch in length in the preferred embodiment. Each node 18 is cylindrical with a generally flat lower surface 20 having rounded corners, or with a slightly rounded lower surface 20, for making comfortable contact with the user's wrist. However, pressure-applying nodes 18 may be sized and shaped dependent upon the needs and desires of the user. Pressure-applying nodes 18 preferably simulate human fingers and make comfortable contact with the user's body.

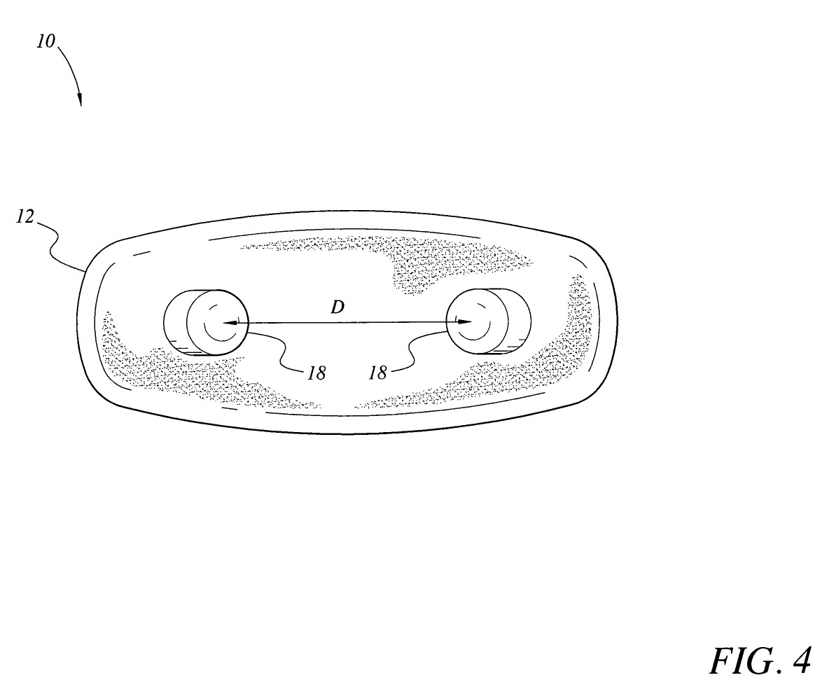

As shown in the bottom view of FIG. 4, pressure-applying nodes 18 are preferably positioned symmetrically about the central axis of gripping handle portion 12 and are spaced apart a distance D. In the preferred embodiment, distance D is approximately 1 ½ to 2 inches. The lower surface of gripping handle portion 12 is shown as having a substantially oval contour, however, the lower surface may be contoured dependent upon the needs and desires of the user.

Returning to FIG. 3, a first recess 24 is formed substantially centrally along the upper end of gripping handle portion 12. Formed in the opposite side face of gripping handle portion 12 is a second recess 16, shown in FIG. 2. When the user grasps gripping handle portion 12, as in FIG. 1, second recess 16 is adapted and contoured for comfortably receiving the user's thumb. Similarly, first recess 24 is adapted and contoured for comfortably receiving at least one of the user's fingers. Additionally, first recess 24 may be contoured to include indentations 25, one such indentation 25 being formed for each finger. Here, indentations 25 of recess 24 are shown as being adapted for receiving three of the user's fingers (the index, middle and ring fingers or fingertips, although first recess 24 may be sized and contoured dependent upon the needs and desires of the user.

In use, the user grasps the gripping handle portion 12 so that the user's thumb rests within second recess 16, and the user's fingers rest within contoured first recess 24. The user positions the tool 10 above either the user's wrist or the wrist of another person, as illustrated in FIG. 1, so that pressure-applying nodes 18 may make gentle contact with the pressure points in the wrist. Pressure-applying nodes 18 are spaced apart so that simultaneous contact of the pressure points is made and so that the user does not require expertise in acupressure to locate the pressure points. Pressure-applying nodes 18 have contact surfaces 20 that provide comfortable contact with the user's wrist, and lower surface 22 is contoured to conform to the user's wrist.

It is to be understood that the present invention is not limited to the embodiment described above, but encompasses any and all embodiments within the scope of the following claims.

We claim:

1. An acupressure tool for relief of lower back pain, comprising:
a gripping handle having an upper surface, a lower surface and opposed first and second side surfaces, the side surfaces being adapted for grasping by a user; and
first and second pressure-applying nodes mounted to the lower surface of the gripping handle and projecting downwardly therefrom, the pressure-applying nodes being spaced apart and adapted for applying pressure to two acupressure points in a person's wrist for relieving lower back pain.

2. The acupressure tool as recited in claim 1, wherein the first side surface has a first recess formed therein for receiving at least one finger of the user's hand.

3. The acupressure tool as recited in claim 2, wherein the second side surface has a second recess formed therein for receiving the thumb of the user's hand.

4. The acupressure tool as recited in claim 2, wherein the first recess includes a plurality of indentations contoured for receiving a plurality of fingers of the user's first hand.

5. The acupressure tool as recited in claim 1, wherein the lower surface of said gripping handle has a substantially arcuate cross-sectional contour adapted for conforming to the ventral aspect of the user's wrist.

6. The acupressure tool as recited in claim 5, wherein said first and second pressure-applying nodes each project along an axis substantially normal to the lower surface of said gripping handle.

7. The acupressure tool as recited in claim 1, wherein each of said first and second pressure-applying nodes has a contact surface for contacting the selected body part of the user, the contact surface having a substantially smooth and rounded contour for making comfortable contact with the person's wrist.

8. The acupressure tool as recited in claim 1, wherein the upper surface of said gripping handle has a substantially arcuate cross-sectional contour.

9. The acupressure tool as recited in claim 1, wherein said gripping handle is made from a transparent plastic material.

10. The acupressure tool as recited in claim 1, wherein said gripping handle is made from a transparent acrylic.

11. The acupressure tool as recited in claim 1, wherein said gripping handle comprises a hollow body.

12. The acupressure tool as recited in claim 1, wherein said pressure-applying nodes are made from natural rubber.

13. The acupressure tool as recited in claim 1, wherein said pressure-applying nodes are made from synthetic rubber.
